(19) United States
(12) Patent Application Publication
(10) Pub. No.: US 2025/0302714 A1
(43) Pub. Date: Oct. 2, 2025

(54) ORAL HEALTH AGENT AND COMPOSITION FOR USE IN DOMESTIC ANIMALS

(71) Applicants: Daniela FISCHER RUSSELL, Victoria (CA); Darcy Edmund BOMFORD, Vernon (CA)

(72) Inventors: Daniela FISCHER RUSSELL, Victoria (CA); Darcy Edmund BOMFORD, Vernon (CA)

(21) Appl. No.: 18/872,986

(22) PCT Filed: Jun. 13, 2023
(86) PCT No.: PCT/CA2023/050809

§ 371 Date: Dec. 9, 2024

Related U.S. Application Data
(60) Provisional application No. 63/351,745, filed on Jun. 13, 2022.

(51) Int. Cl. A61K 8/43; A61K 8/04; A61Q 11/00

(52) U.S. Cl. CPC A61K 8/43; A61K 8/042; A61Q 11/00; A61K 2800/92

(57) ABSTRACT

Provided herein are the use of a specific amino acid, namely, arginine for the prevention and disruption of oral biofilm formation in domestic pets. More specifically, the invention discloses the use, composition and methods for influencing the growth of healthy bacteria and the reduction of disease associated microorganisms in oral biofilm. The compositions include arginine as a biofilm modulator in domestic animals. The invention also provides methods for the prevention and treatment of pathogenic oral biofilm.

This application claims the benefit of U.S. Provisional Application No. 63/351,745 filed Jun. 13, 2022, the contents of which are incorporated herein by reference.

### FIELD

The present disclosure relates to compositions and methods for preventing or combating oral biofilm, dental plaque, oral odor, gingivitis and periodontal disease in non-human animals. In particular, the present disclosure relates to the use of L-arginine and/or similar amino-acids to modify the oral cavity environment and the oral microbiome of domestic animals.

### BACKGROUND

Oral biofilms, commonly known as dental plaque, are complex microbial communities that form on tooth surfaces and other oral structures. These biofilms are primarily composed of bacteria and are responsible for various oral health problems, including dental caries, gingivitis, and periodontal disease. Pets are not as susceptible to cavities but plaque often leads to gingivitis, periodontal disease and tooth decay. Current methods for controlling oral biofilm involve mechanical removal through brushing, professional dental cleaning and to a lesser degree administration of oral health aids such as dental chews. However, these methods often fall short in providing long-term prevention.

Several compounds have been identified to disrupt biofilm formation and bacterial growth. Among them L-arginine showed very promising effect in humans favouring commensal bacteria vs Streptococcus mutans, the causative agent of cavities (Sharma 2014, Nascimento 2009). Arginine, for oral administration, has been vastly studied in humans, mostly due to its ability to in combination with fluoride, reducing dental sensitivity and inducing tooth mineralization.

An expanding area of oral microbiology in humans has now been devoted to explore microbial metabolic activities that help to neutralize biofilm pH and thus inhibit the caries process. Arginine metabolism via the arginine deiminase pathway (ADS) produces alkali in the form of ammonia that counteracts the effects of biofilm acidification from bacterial glycolysis. ADS also functions as an adaptive strategy used by certain bacteria to thrive in oral biofilms.

Most recently, Carda-Dieguez et al. 2022 demonstrated the use of a combination of fluoride and arginine in dentrifice in the modification of the human oral microbiome in humans. Although the body of literature could be viewed as indicative, due to the vast physiological, metabolic and compositional differences of dogs and cats oral environment is not possible to extrapolate human information to pets. Experimentation is required to demonstrate activity of compounds in oral health.

Given the significant differences in the oral microbiome and oral environment of pets when compared to humans, data obtained in humans is not predictive of effectiveness in non-humans such as domestic animals.

Accumulation of periodontal biofilm and plaque formation is commonly seen in dogs of all breeds. Biofilm is known to lead to plaque formation and periodontal disease affecting up to 85% of the canine population.

Dental disease is the result of a multi-step process that occurs within the oral cavity. In the first step, the tooth is coated with a thin layer of glycoproteins commonly known as the acquired pellicle. This layer adheres tightly to the tooth surface and cannot be removed with routine oral care measures such as brushing. Within hours, this layer will thicken as debris and normal oral bacteria accumulate along the gingival margin and begin to adhere and multiply. At this point, the plaque biofilm (bacteria plus secreted matrix) is quite soft and can be removed by simple abrasion from devices such as brushing or chewing. However, with time, the bacterial population will increase and the plaque will become more secured to the tooth surface through the production of glucans.

The presence of a mature plaque biofilm on the tooth surface creates a condition in which mineralization can occur within the bacteria matrix. This process involves the precipitation of salivary components into a deposit commonly known as calculus, or tartar. Calculus is formed when soluble minerals, such as calcium carbonate and calcium phosphate, become supersaturated within the interstitial spaces between the bacteria during conditions of alkaline pH. Mucopolysaccharides, formed by the microorganisms in the plaque, are thought to play a role in this crystallization3. Unlike plaque, once formed, calculus can only be removed via a professional scaling procedure. Further, if left to accumulate, the porous surface of the calculus can serve as a nidus for new plaque bacteria. The accumulation of such bacteria along the gum line can create irritation of the soft tissues and eventually lead to gingival recession or loss of ligament integrity.

Dental plaque biofilms typically contain tens to hundreds of bacterial species. Oral biofilm architecture, species composition, and spatial arrangement of the contained species impact growth-rates and can enhance tolerance to adverse environmental conditions. Depending upon the location (supragingival versus subgingival), the biomass (number of bacteria), the species composition (types and relative abundance), and spatial arrangement of the constituent species (in three dimensions), dental plaque biofilms can cause tooth decay or periodontal disease. Dental plaque biofilm communities are extremely resistant to external chemical and physical insults. For example, they are up to 1,000 times less susceptible to antimicrobials compared to their planktonic counterparts, and are typically resistant to abrasive treatments. This can turn to additional antimicrobial drug resistance induction.

Historically, the most common means to help prevent dental disease was with routine brushing and prophylaxis. Brushing is held by Veterinary health professionals as the single most effective means of reducing biofilm and removing plaque; however, owner compliance is extremely low. This has led to the development of a multitude of products (e.g., dental diets, chews, and toys) with special abrasive textures designed to clean the chewing surfaces of the teeth. Since abrasive products only work on contact surfaces, some companies have tried to offset this limitation by offering antimicrobial compounds incorporated into pet toothpastes, gels, rinses and water supplements such as enzymes (glucose oxidase and lactoperoxidase), chelators (zinc and EDTA combination) and antibiotics (Chlorhexidine). Pet food alternatives were also developed by developing kibbles that can mimic the abrasive action of toothbrushing and coating pet foods with compounds that decrease crystallization of calcium onto the gingival spaces and plaque. That is the case of polyphosphates that have been recently linked to metabolic imbalance leading to nephrotoxicity.

Although many of these treatments can be effective, several of the compounds utilized have been linked to toxicity issues and long-term use is not recommended, as it is the case of chlorhexidine. One of the biggest differences between human and animal consumption of these products is the fact that dogs swallow the product in its entirety creating additional concern upon extended exposure.

Furthermore, several products focus greatly on decreasing plaque by combating secondary mechanisms of accumulation such as mineral deposits instead of controlling microorganisms that cause the issue. Alternatively, antimicrobials can be used to control biofilm development, but they are often non-specific in their activity and also kill the bacteria found in the healthy oral microbiome.

Amino acids such as L-arginine have been shown to help prevent the development of dental biofilms in humans (Koldeman et al., 2015, Nascimento et al., 2014, Huang et al., 2017). Research into the mechanism of action of L-arginine has primarily centered on the ability of oral streptococci to catabolize L-arginine and consequently generate a local pH rise that counteracts the deleterious effects of acid on teeth. Evidence also suggests that, while micromolar concentrations of L-arginine metabolically stabilize bacteria within coaggregates and can mediate cell-cell signaling in dental plaque biofilms, millimolar concentrations can disaggregate bacterial coaggregates and can influence the adhesion of Streptococcus mutans to tooth surfaces.

In humans, L-arginine can play a role in biofilm metabolism and development, as it may destabilize oral multi-species biofilm communities making them more susceptible to antimicrobial treatments. Arginine has also been demonstrated to impact oral health pH, directly due to its basic form or indirectly through its metabolization by bacteria expressing the arginine deiminase system (ADS). In humans, L-Arginine hydrochloride (LAHCI) has been shown to alter the 3-dimensional architecture and biovolume in in vitro model systems of multi-species biofilms and to enhance the antimicrobial efficacy of cetylpyridinium chloride, a compound that shows retarded biofilm penetration.

One strategy to combat oral biofilm, plaque formation and gum disease is to target the oral microbiome. This strategy is based on favouring the growth of certain microorganisms associated with a healthy mouth which in turn create conditions such as pH modifications that retard the growth of microorganisms associated with disease.

Although commonalities exist in the strategy among species, this strategy is heavily dependent on the species-specific microbiome.

Significant differences in oral microbiome exist between humans and domestic such as cats and dogs. These differences are in part due to the species specific physiological characteristics. For example, the oral pH in humans varies between 6.2 and 7.6 while the oral pH in dogs is in the range of 7.5 and 8.0. Dogs also have vastly different oral proteomic composition in their saliva differing in the expression of more than 2500 proteins from humans as identified by Sanguansermsri et al. (2018) using shotgun proteomics, in addition to mineral composition. The differences in the canine and feline oral environment are believed to be responsible for the microbiota composition and increased rate of calcification and plaque formation in dogs and cats.

A dynamic interplay exists between microbiomes and their particular environment with recent demonstration of significant changes in microbiocidal population within hours of stress insult.

This interplay makes it difficult to assert whether the environment dictates the microbiome or vice versa. It is possible that the acidogenic nature of the human oral microbiome is a consequence of selection due to diet including a high consumption of sugars. The human oral microbiome is comprised of several sugar metabolizing microorganisms whose activity lead to acid production. Among these are Streptococcus mutants, the causative agent of dental cavities, the most prevalent oral health issue in humans.

Cavities are not a prevalent issue in dogs which suffer from a much higher prevalence of periodontal disease (80% of dogs over the age of 3 years) and consequent tooth decay. In fact, streptococcus is not a relevant population in dogs' oral microbiome rendering human oral health treatments ineffective for dogs other than broad spectrum antibiotics.

Instead dogs' healthy oral microbiome is composed mostly of gram negative aerobes switching to a gram positive anaerobic population as plaque and periodontal disease develops.

TABLE 1
Most predominant microorganisms consistently found in
dogs oral biome based on health status: healthy mouth,
mild periodontal disease and Periodontal disease

| | Mild Periodontal | Periodontal |
|---|---|---|
| Healthy Mouth | disease | disease |
| Moraxella* | Clostridiales | Clostridiales |
| Bergeyella zoohelicum* | Peptostreptococcaceae | Peptostreptococcaceae |
| Neisseria shayeganii* | Corynebacterium canis | Lachnospiraceae |
| Pasteurella spp | | Actinomyces |
| Capnocytophaga | | Porphyromonas |
| Strenotrophomonas | | canigingivalis |
| Porphyromonas | | |
| canigingivalis | | |

*dominant in supragingival biofilms

Despite several studies individual pathogens could not be correlated with oral disease in dogs or cats making the human in vitro and clinical studies unreliable in the development of oral care products for domestic animals. There remains a need for oral health products developed specifically for domestic animals such as dogs and cats.

### SUMMARY

The present invention addresses the aforementioned need by providing compositions and methods for the modulation of oral biofilm with the purpose of preventing or combating oral disease in companion animals using an L-arginine preparation. L-arginine can act to inhibit selected bacteria attachment, growth and formation of biofilm.

Treatment in concentrations of 0.005% to 10% of an ingestible product including pet food, chew treats, biscuits, cookies, gels, paste and food toppings (liquid, gel or solid) can be used to deliver the desired concentrations of arginine to the oral cavity.

The ingestible product can be a domestic animal food composition optimizing oral microbiome and with that having oral biofilm and dental calculus prevention activity. The arginine may be incorporated in oral health products or top coated dry food, wet food, or food topping product in liquid, gel, paste or power format.

The ingestible product can be a health composition for companion animals having oral biofilm and dental calculus prevention or disruption activity, and in the form of liquids, gels, pastes, sprays, treats, cookies, chewable treats administered before during or after meals.

The ingestible product can also be a domestic animal food or health composition having oral odor control achieved through modifications of the oral microbial environment by the present agent.

The arginine can act as an oral microbiome modifying agent for companion animals. The arginine-containing composition may in addition contain chelators, antibiotics, or other natural products with odor controlling, biofilm controlling or antibacterial activity.

In one aspect, provided herein is a method of preventing or combating oral biofilm and/or an oral biofilm-related disease or condition in a non-human animal, comprising administrating an effective amount of a composition comprising arginine to the non-human animal.

In another aspect, provided herein is a composition comprising arginine for use in preventing or combating oral biofilm and/or an oral biofilm-related disease or condition in a non-human animal.

In yet another aspect, provided herein is a use of a composition comprising arginine to prevent or combat oral biofilm and/or an oral biofilm-related disease or condition in a non-human animal.

In one embodiment, the arginine comprises free base arginine and/or one or more salt forms of arginine. In one embodiment, the arginine is L-arginine.

In one embodiment, the oral biofilm-related disease or condition is dental plague, calculus, oral odor, gingivitis and/or periodontal disease.

In one embodiment, the composition modulates the oral microbiome composition of the non-human animal.

In one embodiment, the non-human animal is a domestic animal such as a cat or a dog.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a graph showing relative abundance of Moraxella at day 28 in treated vs control animals. Each bar represents an animal.

FIG. 2 is a graph showing relative abundance of Mycobacteriales at day 28 in treated vs control animals. Each bar represents an animal.

FIG. 3 is a graph showing relative abundance of Burkholderiales at day 28 in treated vs control animals. Each bar represents an animal.

FIG. 4 is a graph showing relative abundance of Pasteurella at day 28 in treated vs control animals. Each bar represents an animal.

FIG. 5 is a graph showing relative abundance of Peptoniphilus at day 28 in treated vs control animals. Each bar represents an animal.

FIG. 6 is a graph showing relative abundance of Capnocytophaga at day 28 in treated vs control animals. Each bar represents an animal.

FIG. 7 is a graph showing relative abundance of Clostridia at day 28 in treated vs control animals. Each bar represents an animal.

FIG. 8 is a graph showing relative abundance of Lautropia at day 28 in treated vs control animals. Each bar represents an animal.

FIG. 9 is a graph showing relative abundance of Microbacteriaceae at day 28 in treated vs control animals. Each bar represents an animal.

FIG. 10 is a graph showing relative abundance of all unclassified bacteria at day 28 in treated vs control animals. Each bar represents an animal.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 20%, 5-20%, or 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary.

### II. Methods, Uses and Compositions for Use

The present disclosure provides the use of an amino acid agent for modulating the oral microbiome toward a more beneficial composition ultimately improving oral health conditions in companion animals. The present invention is based on arginine applied as coating or included in food products, feed, treats, chews, gels, pastes and food toppings. This invention is greatly superior to current products as it is of natural origin and shows a great degree of safety much desirable by consumers. Without wishing to be bound by a theory, arginine can act through several mechanisms, among them, the modification of the oral pH and stimulation of bacteria that are associated with a healthy oral state as opposed to other treatments that focus on chelation, sequestration of calcium or broad spectrum bacterial control.

It is demonstrated herein that oral administration of arginine is associated with a shift towards healthy oral bacterial populations in dogs.

Accordingly, in one aspect, provided herein is a method of preventing or combating oral biofilm and/or an oral biofilm-related disease or condition, comprising administrating an effective amount of a composition comprising arginine to the non-human animal.

In another aspect, provided herein is a composition comprising arginine for use in preventing or combating oral biofilm and/or an oral biofilm-related disease or condition.

In yet another aspect, provided herein is a use of a composition comprising arginine to prevent or combat oral biofilm and/or an oral biofilm-related disease or condition.

Examples of oral biofilm-related disease or condition include but are not limited to dental plague, calculus, oral odor, gingivitis and periodontal disease.

In an embodiment, the oral biofilm-related disease or condition is dental plague. In an embodiment, the oral biofilm-related disease or condition is calculus. In an embodiment, the oral biofilm-related disease or condition is oral odor. In an embodiment, the oral biofilm-related disease or condition is gingivitis. In an embodiment, the oral biofilm-related disease or condition is periodontal disease.

The method and uses disclosed herein can be used in a non-human animal, including a domestic animal such as a dog or a cat.

In an embodiment, the non-human animal is a domestic animal. In an embodiment, the domestic animal is a dog. In an embodiment, the domestic animal is a cat.

The term “preventing” or “prevent” as used herein means reducing and/or eliminating the risk or probability of the non-human animal developing oral biofilm and/or biofilm-related disease/condition and/or manifesting a symptom associated with the disease/condition.

The term “combating” or “combat” as used herein means reducing and/or eliminating oral film, reducing the severity of the biofilm-related disease/condition and/or symptoms thereof, delaying or slowing progression of the biofilm-related disease/condition, stabilizing the biofilm-related disease/condition and/or symptoms thereof, delaying onset of the biofilm-related disease/condition etc.

The term “effective amount” as used herein refers to an amount that is effective, at dosages and for periods of time necessary to achieve the desired result. For example, an effective amount can be an amount of arginine that effects a shift in the oral microbiome towards populations associated with healthy oral cavities in a non-human animal.

In an embodiment, the composition modulates the oral microbiome composition of the non-human animal.

The composition can for example increase the relative abundance of one or more microorganisms associated with a healthy oral microbiome and/or decrease the relative abundance of one or more microorganisms associated with periodontal disease.

For example, the composition can increase the relative abundance of one or more of Moraxella, Mycobacteriales, Burkholderiales, Pasteurella, Capnocytophaga, Clostridia, Lautropia and Microbacteriaceae and/or decrease the relative abundance of Peptoniphilus in the oral microbiome of the non-human animal.

In an embodiment, the composition increases the relative abundance of one or more of Burkholderiales, Capnocytophaga and Moraxella in the oral microbiome of the non-human animal.

In an embodiment, the composition decreases the relative abundance of Peptoniphilus in the oral microbiome of the non-human animal.

In an embodiment, the composition increases the relative abundance of one or more of Burkholderiales, Capnocytophaga and Moraxella and/or decreases the relative abundance of Peptoniphilus in the oral microbiome of the non-human animal.

The method disclosed herein comprise administrating a composition comprising arginine. The arginine can be a free base or in a salt form. Examples of suitable salt forms include but are not limited to arginine hydrochloride and arginine bicarbonate.

In an embodiment, the arginine the arginine comprises free base arginine and/or one or more salt forms of arginine. In an embodiment, the one or more salt forms of arginine is arginine hydrochloride.

In an embodiment, the arginine is L-arginine.

In an embodiment, the composition comprises from about 0.5% w/w to about 10% w/w of arginine. In an embodiment, the composition comprises from about 0.5% w/w to about 8% w/w of arginine. In an embodiment, the composition comprises from about 0.8% w/w to about 5% w/w of arginine. In an embodiment, the composition comprises from about 1% w/w to about 2% w/w of arginine.

In an embodiment, the composition comprises about 1.5% w/w arginine.

The composition can be formulated into any suitable form for oral administration, including but not limited to solid, powder, gel, paste, liquid, and spray. Where the non-human animal is a domestic animal, the composition can be formulated into for example a pet food, a pet treat, or a food topping.

The composition may comprise one or more orally acceptable carriers. An “orally acceptable carrier” refers broadly to any component that acts as a carrier, diluent or filler. Any suitable orally acceptable carrier may be used, depending on the intended formulation. For example, if the composition is to be formulated into a gel, then the composition may comprise water as an orally acceptable carrier. If the composition is to be formulated into a pet food, then the composition may comprise food ingredients such as wheat flour as orally acceptable carriers. It is understood that an orally acceptable carrier is generally safe for consumption and non-toxic to the non-human animal.

In an embodiment, the composition further comprises one or more orally acceptable carriers.

The composition may further comprise one or more agents for one or more purposes. For example, the composition may comprise a flavouring agent, such as a sweetener, to enhance the palatability of the composition. For example, the composition may comprise a texturizing agent to achieve a desired texture for the intended non-human animal.

The composition may further comprise chelators, antibiotics, and/or other natural products with odor controlling, biofilm controlling and/or antibacterial activities.

The composition may be administered during a meal. For example, the composition may be incorporated into or on a food, or it may be formulated as a food topping.

The composition may be administered before or after a meal. For example, the composition may be incorporated into or on a treat.

The composition may also be formulated for direct administration to the oral cavity of the non-human animal. For example, the composition may be formulated as a mouth gel, a mouth rinse, a mouth spray or a water additive.

Although any methods and uses similar or equivalent to those described herein can also be used in the practice or testing of the present disclosure, examples of methods and uses are now described.

### EXAMPLES

### Example 1: Clinical Study

While it is reasonable to anticipate that the dog oral biofilm can potentially be modulated by arginine, the drastic differences in oral characteristics and microbiome composition make this assumption questionable. This cannot be asserted without undue experimentation.

In vitro and in vivo studies were required to assert the activity of arginine in oral care of domestic animals. In the present disclosure the activity of arginine in the modulation of the canine oral microbiome is demonstrated. Controlled clinical studies were performed in 14 beagles fed an arginine powder mixture (Table 2) twice daily after meals. Dental assessments were performed using photographic inspection after the application of a biofilm disclosing agent. Animals were fed the mixture for 28 days prior to final assessment. Oral swab samples were collected at days 0, 7, 14 and 28 and subjected to global sequencing studies.

TABLE 2
Ingredients of arginine powder mixture

| | |
|---|---|
| Oat flour | 89% |
| Calcium carbonate | 1.5% |
| Calcium Phosphate | 1.5% |
| L-arginine | 1.5% |
| Canola oil | 2% |
| AFB palatant | 3% |
| Sea salt | 0.5% |
| Antioxidant | 0.1% |
| Oil of Oregano | 0.09% |
| Thyme Oil | 0.04% |

This study identified the effects of arginine on the canine oral microbiome for the first time.

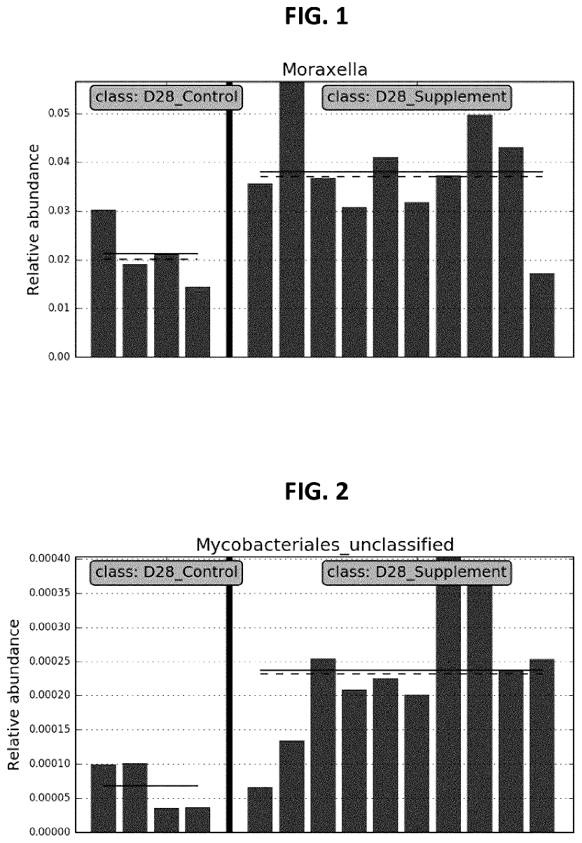

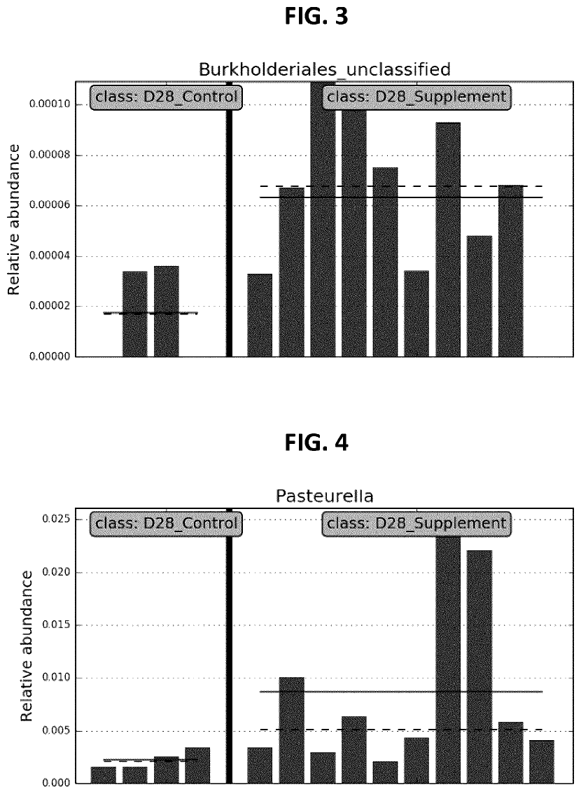

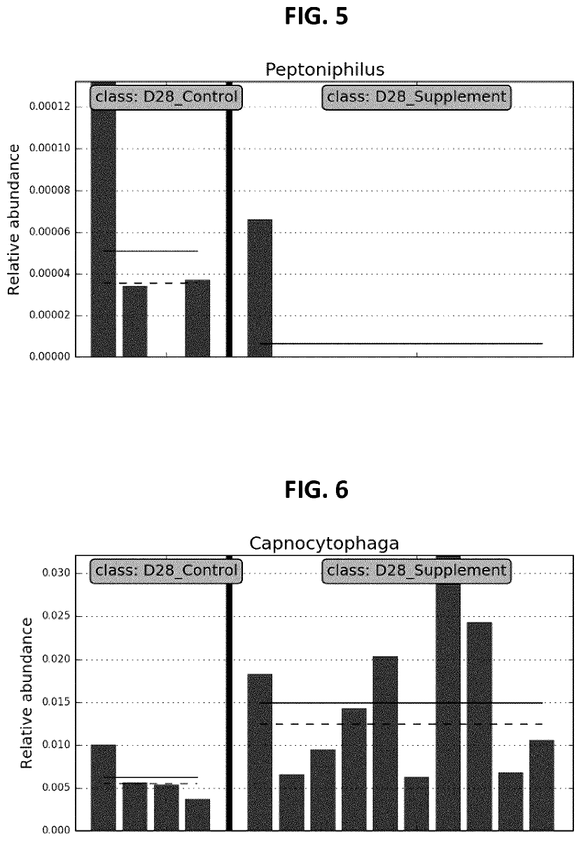

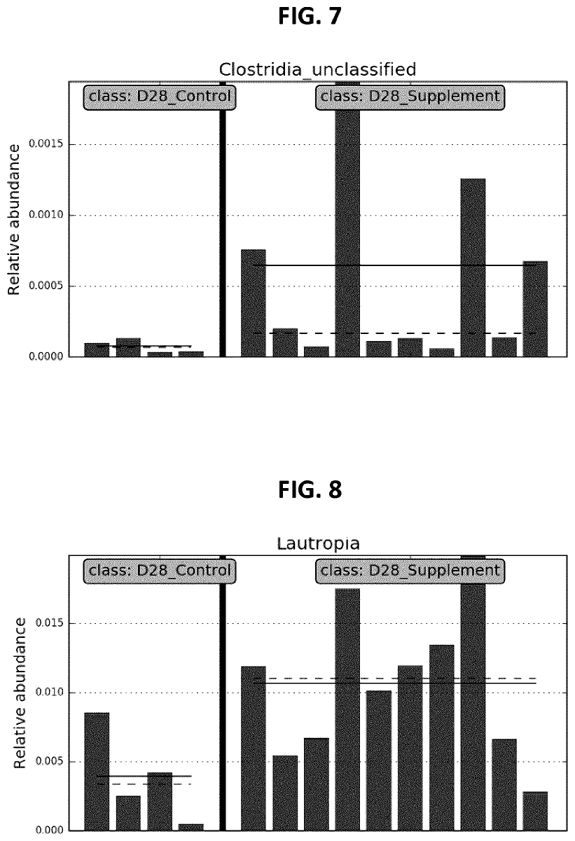

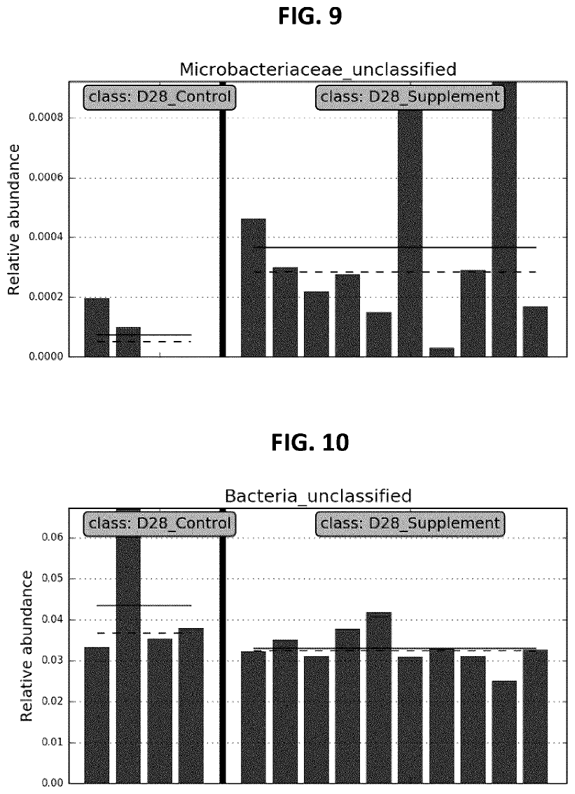

In this study bacterial populations were clustered and plotted in separate figures. Data was obtained after 28 days of treatment post dental cleaning. Each graph in FIGS. 1-10 shows a comparison of the relative abundance of each microorganism order between treated and control animals.

These data demonstrates a shift towards “healthy” bacteria populations including Burkholderiales, capnocytophaga and Moraxella.

### Example 2: Food Composition for the Modulation of Oral Microbiome Favouring Bacteria Associated with a Healthy State

Food ingredients including flours, starch, coconut oil, flavouring agents, and 1.5% L-arginine are mixed thoroughly until a homogenous solution is obtained. The derived product is processed by hot or cold extrusion and manufactured in the form of pet treats.

### Example 3: Method of Improving Oral Microbiome Through the Administration of Oral Rinses or Gels

To favour a healthy oral microbiome and reduce biofilm, L-arginine is administered as a mouth gel, rinse or water additive to domestic animals. Pet owners are instructed to provide composition in water two to three times daily or apply gel or paste directly to the teeth of the animals.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

The scope of the claims should not be limited by the preferred embodiments and examples, but should be given the broadest interpretation consistent with the description as a whole.

### CITATIONS FOR REFERENCES REFERRED TO IN THE SPECIFICATION

Clarke, D. E., Servet, E., Hendriks, W. et al (2010) Effect of kibble size, shape and additives on plaque in cats. J Vet Dent 27:84-90.
Dobenecker B, Reese S, Herbst S (2021) Effects of dietary phosphates from organic and inorganic sources on parameters of phosphorus homeostasis in healthy adult dogs. PLOS ONE 16(2): e0246950.
Gilbert P, Maira-Litran T, McBain A J, Rickard A H, Whyte F W. The physiology and collective recalcitrance of microbial biofilm communities. Adv Microb Physiol. 2002; 46:202-256.
Hennet, P., Servet, E., Soulard, Y. and Biourge, V. (2007) Effect of pellets size and polyphosphates in preventing calculus accumulation in dogs. J Vet Dent 24:236-239.
Hojo K, Nagaoka S, Ohshima T, Maeda N. Bacterial interactions in dental biofilm development. J Dent Res. 2009; 88:982-990.
Kolderman E, Bettampadi D, Samarian D, Dowd S E, Foxman B, et al. (2015) L-Arginine Destabilizes Oral Multi-Species Biofilm Communities Developed in Human Saliva. PLOS ONE 10(5): e0121835.
Kolenbrander P E, Palmer R J Jr., Rickard A H, Jakubovics N S, Chalmers N I, Diaz P I. Bacterial interactions and successions during plaque development. Periodontol 2000. 2006; 42:47-79.
Larmas M, Scheinin A. Studies on dog saliva: I. Some physico-chemical characteristics. Acta Odontol Scandi. 1971; 29:205-14.
Marsh P D. Controlling the oral biofilm with antimicrobials. J Dent. 2010; 38 Suppl 1: S11-15.
Nascimento M M, Gordan V V, Garvan C W, Browngardt C M, Burne R A. Correlations of oral bacterial arginine and urea catabolismwith caries experience. Oral Microbiol Immunol. 2009; 24:89-95.
Nascimento M M, Liu Y, Kalra R, Perry S, Adewumi A, Xu X, et al. Oral arginine metabolism may decrease the risk for dental caries in children. J Dent Res. 2013; 92:604-608.
Rogers A H, Zilm P S, Gully N J. Influence of arginine on the coexistence of Streptococcus mutans and S. milleri in glucose-limited mixed continuous culture. Microb Ecol. 1987; 14:193-202.
Sanguansermsri P, Jenkinson H F, Thanasak J, Chairatvit K, Roytrakul S, Kittisenachai S, Puengsurin D, Surarit R. Comparative proteomic study of dog and human saliva. PLOS One. 2018 Dec. 4; 13 (12): e0208317. doi: 10.1371/journal.pone.0208317.
Sharma S, Lavender S, Woo J, Guo L, Shi W, Kilpatrick-Liverman L, et al. Nanoscale characterization of effect of L-arginine on Streptococcus mutans biofilm adhesion by atomic force microscopy. Microbiology. 2014; 160:1466-1473. pmid: 24763427
Stookey G K, Warrick J M, Miller L L. Effect of sodium hexametaphosphate on dental calculus formation in dogs. Am J Vet Res. 1995 July; 56 (7): 913-8.
Huang, X., Zhang, K., Deng, M., Exterkate, R. A., Liu, C., Zhou, X., Cheng, L. and Ten Cate, J. M., 2017. Effect of arginine on the growth and biofilm formation of oral bacteria. Archives of oral biology, 82, pp. 256-262.
Zijnge V, van Leeuwen M B, Degener J E, Abbas F, Thurnheer T, Gmür R, et al. Oral biofilm architecture on natural teeth. PloS One. 2010; 5: e9321.

1.-21. (canceled)

22. A method of preventing or combatting oral biofilm and/or an oral biofilm-related disease or condition in a non-human animal comprising administering a composition comprising arginine to the non-human animal.

23. The method of claim 22, wherein the arginine comprises free base arginine and/or one or more salt forms of arginine.

24. The method of claim 23, wherein the one of more salt forms of arginine is arginine hydrochloride.

25. The method of claim 22, wherein the arginine is L-arginine.

26. The method of claim 22, wherein the oral biofilm-related disease or condition is dental plague, calculus, oral odor, gingivitis and/or periodontal disease.

27. The method of claim 22, wherein the composition modulates the oral microbiome composition of the non-human animal.

28. The method of claim 22, wherein the composition increases the relative abundance of one of more of Moraxella, Mycobacteriales, Burkholderiales, Pasteurella, Capnocytophaga, Clostridia, Lautropia and Microbacteriaceae in the oral microbiome of the non-human animal.

29. The method of claim 22, wherein the composition increases the relative abundance of one of more of Burkholderiales, Capnocytophaga and Moraxella in the oral microbiome of the non-human animal

30. The method of claim 22, wherein the composition decreases Peptoniphilus in the oral microbiome of the non-human animal.

31. The method of claim 22, wherein the composition comprises from about 0.5% w/w to about 10% w/w of arginine, or from about 0.5% w/w to about 8% w/w of arginine, or from about 0.8% w/w to about 5% w/w of arginine, or from about 1% w/w to about 2% w/w of arginine.

32. The method of claim 22, wherein the composition comprises about 1.5% w/w of arginine.

33. The method of claim 22, wherein the non-human animal is a dog or a cat.

34. The method of claim 22, wherein the composition is formulated as a solid, a powder, a gel, a paste, a liquid, or a spray.

35. The method of claim 22, wherein the composition is formulated as a food, a food topping, or a treat.

36. The method of claim 35, wherein the treat is a chewable treat.

37. The method of any one of claim 22, wherein the composition is administered during a meal.

38. The method of claim 22, wherein the composition is administered before or after a meal.

39. The method of claim 22, wherein the composition is formulated as a mouth gel, a rinse, a spray or a water additive.

40. The method of claim 39, wherein the composition is administered directly to the teeth of the non-human animal.

41. The method of claim 22, wherein the composition further comprises an orally acceptable carrier.
